(19) United States
(12) Patent Application Publication
(10) Pub. No.: US 2024/0285951 A1
(43) Pub. Date: Aug. 29, 2024

(54) PERSONALIZED NEUROSTIMULATION CLOSED LOOP PROGRAMMING TRAINING INTERFACE

(71) Applicant: Boston Scientific Neuromodulation Corporation, Valencia, CA (US)

(72) Inventors: Dat Thanh Huynh, North Hollywood, CA (US); Matthew Lee McDonald, Glendale, CA (US); Bradley Lawrence Hershey, Carrollton, TX (US); Kristen Marie Lechleiter, Davidson, NC (US)

(21) Appl. No.: 18/586,346

(22) Filed: Feb. 23, 2024

Related U.S. Application Data
(60) Provisional application No. 63/448,115, filed on Feb. 24, 2023.

(51) Int. Cl. A61N 1/36; A61N 1/02; A61N 1/372; G06N 20/00; G16H 40/40

(52) U.S. Cl. CPC A61N 1/36132; A61N 1/025; A61N 1/37247; G06N 20/00; G16H 40/40

(57) ABSTRACT

A system to analyze data for neurostimulation programming may include capabilities to collect and analyze relevant input data (training data) for training a neurostimulation programming model. In an example, the system may: determine usage of a particular neurostimulation treatment, for a neurostimulation treatment that is associated with a set of parameters used by a neurostimulation device to deliver neurostimulation to a patient; evaluate patient feedback data (e.g., obtained from the patient) associated with the usage of the neurostimulation treatment; calculate, based on the patient feedback data, an amount of training data required to perform training operations on a programming selection model, with such training data being provided at least in part from the patient feedback data and optionally device data. In a further example, the training operations on the programming selection model enable an updated set of parameters to be generated for the neurostimulation device, such as with closed-loop programming.

### CLAIM OF PRIORITY

This application claims the benefit of U.S. Provisional Application No. 63/448,115 filed on Feb. 24, 2023, which is hereby incorporated by reference in its entirety.

### TECHNICAL FIELD

This document relates generally to data processing obtained in connection with the use of medical devices, and more particularly, to systems, devices, and methods for obtaining data in connection with implanted electrical stimulation, including user input data related to neurostimulation treatments used for pain treatment, movement disorders, and/or management of such conditions.

### BACKGROUND

Neurostimulation, also referred to as neuromodulation, has been proposed as a therapy for a number of conditions. Examples of neurostimulation include Spinal Cord Stimulation (SCS), Deep Brain Stimulation (DBS), Peripheral Nerve Stimulation (PNS), and Functional Electrical Stimulation (FES). A neurostimulation system can be used to electrically stimulate tissue or nerve centers to treat nervous or muscular disorders. For example, an SCS system may be configured to deliver electrical pulses to a specified region of a patient's spinal cord, such as particular spinal nerve roots or nerve bundles, to produce an analgesic effect that masks pain sensation, or to produce a functional effect that allows increased movement or activity of the patient. Other forms of neurostimulation may include a DBS system which uses similar pulses of electricity at particular locations in the brain to reduce symptoms of essential tremors, Parkinson's disease, psychological disorders, or the like.

Various approaches are being developed to enable personalized programming and optimized forms of programming used by neurostimulation systems, including partially or fully automated forms of generating or delivering specific neurostimulation parameters known as closed-loop programming. Some closed-loop programming approaches can, for example, customize changes to stimulator programs, such as to suggest or automatically select a program when a particular condition is identified. However, closed-loop programming typically requires a robust and extensive set of inputs, to accurately learn characteristics about the individual patient and to adequately train a model for implementing the programming.

### SUMMARY

An example (e.g., “Example 1”) is a system to analyze data for neurostimulation programming, the system comprising: one or more processors; and one or more memory devices comprising instructions, which when executed by the one or more processors, cause the one or more processors to: determine usage of a particular neurostimulation treatment, wherein the neurostimulation treatment is associated with a set of parameters used by a neurostimulation device to deliver neurostimulation to a human patient; evaluate patient feedback data associated with the usage of the neurostimulation treatment, wherein the patient feedback data is obtained from the human patient; and calculate, based on the patient feedback data, an amount of training data required to perform training operations on a programming selection model, wherein the training data is provided at least in part from the patient feedback data, and wherein the training operations on the programming selection model enable an updated set of parameters for the neurostimulation device.

In Example 2, the subject matter of Example 1 optionally includes subject matter where the instructions further cause the one or more processors to: generate a user interface display to include one or more metrics that correspond to the amount of training data required to perform the training operations on the programming selection model.

In Example 3, the subject matter of Example 2 optionally includes subject matter where the user interface display further includes one or more metrics that correspond to an amount of training data available to perform the training operations on the programming selection model, and one or more values that correspond to information missing from the training data required to perform the training operations.

In Example 4, the subject matter of any one or more of Examples 2-3 optionally includes subject matter where the instructions further cause the one or more processors to: generate one or more recommendations to obtain additional portions of the training data required to perform the training operations; and output the one or more recommendations in the user interface display.

In Example 5, the subject matter of Example 4 optionally includes subject matter where the one or more recommendations includes one or more of: one or more recommendations for selection of one or more neurostimulation programs; or one or more recommendations for an amount of usage of one or more neurostimulation programs.

In Example 6, the subject matter of any one or more of Examples 1-5 optionally includes subject matter where the instructions further cause the one or more processors to: evaluate device data associated with the usage of a particular neurostimulation program used to provide the neurostimulation treatment, wherein the device data is obtained from a wearable device or from the neurostimulation device, and wherein the training data is further provided from the device data; wherein operations to calculate the amount of training data required to perform the training operations are further based on the device data.

In Example 7, the subject matter of Example 6 optionally includes subject matter where operations to determine the usage of the neurostimulation treatment are further based on evaluating program usage as indicated by the device data that is obtained from the wearable device or from the neurostimulation device.

In Example 8, the subject matter of any one or more of Examples 1-7 optionally includes subject matter where the patient feedback data relates to one or more of: sleep, pain, movement, fatigue or restfulness, alertness, emotional state, medication state, mobility, or activity, in connection with use of the neurostimulation treatment, and wherein the patient feedback data is obtained from the human patient using one or more: questionnaires, surveys, text entries, or voice inputs.

In Example 9, the subject matter of any one or more of Examples 1-8 optionally includes subject matter where the instructions further cause the one or more processors to: identify, based on the calculated amount of training data required to perform training operations, availability for retraining of the programming selection model; and generate a user interface display that includes a user-selectable option to cause the retraining of the programming selection model.

In Example 10, the subject matter of any one or more of Examples 1-9 optionally includes subject matter where the instructions further cause the one or more processors to: determine usage of multiple neurostimulation programs, wherein the multiple neurostimulation programs are associated with respective sets of parameters; wherein to evaluate the patient feedback data includes to evaluate the usage of each of the multiple neurostimulation programs and the patient feedback data associated with each of the multiple neurostimulation programs; and wherein to calculate the amount of training data required to perform training operations on the programming selection model is further based on the usage of each of the multiple neurostimulation programs and the patient feedback data associated with each of the multiple neurostimulation programs.

In Example 11, the subject matter of any one or more of Examples 1-10 optionally includes subject matter where the training operations on the programming selection model are performed at a remote data service, and wherein the updated set of parameters produced by the training operations are generated by the programming selection model with one or more one of: a new program for the neurostimulation device; a modified program for the neurostimulation device; or a modified schedule for use of multiple programs on the neurostimulation device.

In Example 12, the subject matter of any one or more of Examples 1-11 optionally includes subject matter where the programming selection model is used to provide closed-loop programming for the neurostimulation device, and wherein the programming selection model is trained at a remote data service based on the training data.

In Example 13, the subject matter of Example 12 optionally includes subject matter where the closed-loop programming causes a change to neurostimulation programming settings on the neurostimulation device, and wherein the change to the neurostimulation programming settings controls one or more of: pulse patterns, pulse shapes, a spatial location of pulses, waveform shapes, or a spatial location of waveform shapes, of modulated energy provided with a plurality of leads of the neurostimulation device.

Example 14 is a machine-readable medium including instructions, which when executed by a machine, cause the machine to perform the operations of the system of any of the Examples 1 to 13.

Example 15 is a method to perform the operations of the system of any of the Examples 1 to 13.

Example 16 is a device to analyze data for neurostimulation programming, the device comprising: one or more processors; and one or more memory devices comprising instructions, which when executed by the one or more processors, cause the one or more processors to: determine usage of a particular neurostimulation treatment, wherein the neurostimulation treatment is associated with a set of parameters used by a neurostimulation device to deliver neurostimulation to a human patient; evaluate patient feedback data associated with the usage of the neurostimulation treatment, wherein the patient feedback data is obtained from the human patient; and calculate, based on the patient feedback data, an amount of training data required to perform training operations on a programming selection model, wherein the training data is provided at least in part from the patient feedback data, and wherein the training operations on the programming selection model enable an updated set of parameters for the neurostimulation device.

In Example 17, the subject matter of Example 16 optionally includes subject matter where the instructions further cause the one or more processors to: generate a user interface display to include one or more metrics that correspond to the amount of training data required to perform the training operations on the programming selection model.

In Example 18, the subject matter of Example 17 optionally includes subject matter where the user interface display further includes one or more metrics that correspond to an amount of training data available to perform the training operations on the programming selection model, and one or more values that correspond to information missing from the training data required to perform the training operations.

In Example 19, the subject matter of any one or more of Examples 17-18 optionally includes subject matter where the instructions further cause the one or more processors to: generate one or more recommendations to obtain additional portions of the training data required to perform the training operations; and output the one or more recommendations in the user interface display.

In Example 20, the subject matter of Example 19 optionally includes subject matter where the one or more recommendations includes one or more of: one or more recommendations for selection of one or more neurostimulation programs; or one or more recommendations for an amount of usage of one or more neurostimulation programs.

In Example 21, the subject matter of any one or more of Examples 16-20 optionally includes subject matter where the instructions further cause the one or more processors to: evaluate device data associated with the usage of a particular neurostimulation program used to provide the neurostimulation treatment, wherein the device data is obtained from a wearable device or from the neurostimulation device, and wherein the training data is further provided from the device data; wherein operations to calculate the amount of training data required to perform the training operations are further based on the device data; and wherein operations to determine the usage of the neurostimulation treatment are further based on evaluating program usage as indicated by the device data.

In Example 22, the subject matter of Example 21 optionally includes subject matter where the patient feedback data relates to one or more of: sleep, pain, movement, fatigue or restfulness, alertness, emotional state, medication state, mobility, or activity, in connection with use of the neurostimulation treatment, and wherein the patient feedback data is obtained from the human patient using one or more: questionnaires, surveys, text entries, or voice inputs.

In Example 23, the subject matter of any one or more of Examples 16-22 optionally includes subject matter where the instructions further cause the one or more processors to: identify, based on the calculated amount of training data required to perform training operations, availability for retraining of the programming selection model; and generate a user interface display that includes a user-selectable option to cause the retraining of the programming selection model.

In Example 24, the subject matter of any one or more of Examples 16-23 optionally includes subject matter where the instructions further cause the one or more processors to: determine usage of multiple neurostimulation programs, wherein the multiple neurostimulation programs are associated with respective sets of parameters; wherein to evaluate the patient feedback data includes to evaluate the usage of each of the multiple neurostimulation programs and the patient feedback data associated with each of the multiple neurostimulation programs; and wherein to calculate the amount of training data required to perform training operations on the programming selection model is further based on the usage of each of the multiple neurostimulation programs and the patient feedback data associated with each of the multiple neurostimulation programs.

In Example 25, the subject matter of any one or more of Examples 16-24 optionally includes subject matter where the programming selection model is used to provide closed-loop programming for the neurostimulation device; wherein the closed-loop programming causes a change to neurostimulation programming settings on the neurostimulation device; wherein the change to the neurostimulation programming settings controls one or more of: pulse patterns, pulse shapes, a spatial location of pulses, waveform shapes, or a spatial location of waveform shapes, of modulated energy provided with a plurality of leads of the neurostimulation device; and wherein the programming selection model is trained at a remote data service based on the training data.

Example 26 is a method for analyzing data for neurostimulation programming, comprising: determining usage of a particular neurostimulation treatment, wherein the neurostimulation treatment is associated with a set of parameters used by a neurostimulation device to deliver neurostimulation to a human patient; evaluating patient feedback data associated with the usage of the neurostimulation treatment, wherein the patient feedback data is obtained from the human patient; and calculating, based on the patient feedback data, an amount of training data required to perform training operations on a programming selection model, wherein the training data is provided at least in part from the patient feedback data, and wherein the training operations on the programming selection model enable an updated set of parameters for the neurostimulation device.

In Example 27, the subject matter of Example 26 optionally includes generating a user interface display to include one or more metrics that correspond to the amount of training data required to perform the training operations on the programming selection model.

In Example 28, the subject matter of Example 27 optionally includes subject matter where the user interface display further includes one or more metrics that correspond to an amount of training data available to perform the training operations on the programming selection model, and one or more values that correspond to information missing from the training data required to perform the training operations.

In Example 29, the subject matter of any one or more of Examples 27-28 optionally include generating one or more recommendations to obtain additional portions of the training data required to perform the training operations; and outputting the one or more recommendations in the user interface display.

In Example 30, the subject matter of Example 29 optionally includes subject matter where the one or more recommendations includes one or more of: one or more recommendations for selection of one or more neurostimulation programs; or one or more recommendations for an amount of usage of one or more neurostimulation programs.

In Example 31, the subject matter of any one or more of Examples 26-30 optionally include evaluating device data associated with the usage of a particular neurostimulation program used to provide the neurostimulation treatment, wherein the device data is obtained from a wearable device or from the neurostimulation device, and wherein the training data is further provided from the device data; wherein calculating the amount of training data required to perform the training operations are further based on the device data; and wherein determining the usage of the neurostimulation treatment are further based on evaluating program usage as indicated by the device data.

In Example 32, the subject matter of Example 31 optionally includes subject matter where the patient feedback data relates to one or more of: sleep, pain, movement, fatigue or restfulness, alertness, emotional state, medication state, mobility, or activity, in connection with use of the neurostimulation treatment, and wherein the patient feedback data is obtained from the human patient using one or more: questionnaires, surveys, text entries, or voice inputs.

In Example 33, the subject matter of any one or more of Examples 26-32 optionally include identifying, based on the calculated amount of training data required to perform training operations, availability for retraining of the programming selection model; and generating a user interface display that includes a user-selectable option to cause the retraining of the programming selection model.

In Example 34, the subject matter of any one or more of Examples 26-33 optionally include determining usage of multiple neurostimulation programs, wherein the multiple neurostimulation programs are associated with respective sets of parameters; wherein evaluating the patient feedback data includes evaluating the usage of each of the multiple neurostimulation programs and the patient feedback data associated with each of the multiple neurostimulation programs; and wherein calculating the amount of training data required to perform training operations on the programming selection model is further based on the usage of each of the multiple neurostimulation programs and the patient feedback data associated with each of the multiple neurostimulation programs.

In Example 35, the subject matter of any one or more of Examples 26-34 optionally includes subject matter where the programming selection model is used to provide closed-loop programming for the neurostimulation device; wherein the closed-loop programming causes a change to neurostimulation programming settings on the neurostimulation device; wherein the change to the neurostimulation programming settings controls one or more of: pulse patterns, pulse shapes, a spatial location of pulses, waveform shapes, or a spatial location of waveform shapes, of modulated energy provided with a plurality of leads of the neurostimulation device; and wherein the programming selection model is trained at a remote data service based on the training data.

This Summary is an overview of some of the teachings of the present application and not intended to be an exclusive or exhaustive treatment of the present subject matter. Further details about the present subject matter are found in the detailed description and appended claims. Other aspects of the disclosure will be apparent to persons skilled in the art upon reading and understanding the following detailed description and viewing the drawings that form a part thereof, each of which are not to be taken in a limiting sense. The scope of the present disclosure is defined by the appended claims and their legal equivalents.

### BRIEF DESCRIPTION OF THE DRAWINGS

Various embodiments are illustrated by way of example in the figures of the accompanying drawings. Such embodiments are demonstrative and not intended to be exhaustive or exclusive embodiments of the present subject matter.

FIG. 1 illustrates a data processing system to collect and analyze healthcare-related data in connection with the use of neurostimulation programs and closed-loop neurostimulation programming, according to an example.

FIG. 2 illustrates a neuromodulation system as an example of a medical device system, according to an example.

FIG. 3 further illustrates a neuromodulation system as an ambulatory medical device, such as implemented by a neurostimulation treatment, according to an example.

FIG. 4 illustrates a neuromodulation device connected to a programming device, according to an example.

FIG. 5 illustrates data interactions with a closed loop programming data service, for operation of a neuromodulation device with closed loop programming, according to an example.

FIG. 6 depicts a workflow for closed-loop programming of a neuromodulation system, according to an example.

FIG. 7A and FIG. 7B depict data input user interfaces provided in connection with a collection of training data for closed-loop programming, according to an example.

FIG. 8A, FIG. 8B, FIG. 8C, and FIG. 8D depict data output user interfaces provided in connection with a collection of training data for closed-loop programming, according to an example.

FIG. 9 illustrates data types and processing logic associated with data input or output examples of closed-loop programming, according to an example.

FIG. 10 illustrates a data processing flow for updating a neurostimulation treatment of a human patient, including in response to closed-loop programming operations, according to an example.

FIG. 11 illustrates a processing method implemented by a system or device to analyze data, including for training data for a programming model used in closed-loop programming operations, according to an example.

FIG. 12 illustrates, by way of example, a block diagram of an embodiment of a system (e.g., a computing system) for performing patient data analysis in connection with closed-loop programming operations, according to an example.

FIG. 13 illustrates, by way of example, a block diagram of an embodiment of a system (e.g., a computing system) implementing neurostimulation programming circuitry to cause programming of an implantable electrical neurostimulation device, according to an example.

FIG. 14 is a block diagram illustrating a machine in the example form of a computer system, within which a set or sequence of instructions may be executed to cause the machine to perform any one of the methodologies discussed herein, according to an example.

### DETAILED DESCRIPTION

The following detailed description of the present subject matter refers to the accompanying drawings which show, by way of illustration, specific aspects and embodiments in which the present subject matter may be practiced. These embodiments are described in sufficient detail to enable those skilled in the art to practice the present subject matter. Other embodiments may be utilized and structural, logical, and electrical changes may be made without departing from the scope of the present subject matter. References to “an”, “one”, or “various” embodiments in this disclosure are not necessarily to the same embodiment, and such references contemplate more than one embodiment. The following detailed description is, therefore, not to be taken in a limiting sense, and the scope is defined only by the appended claims, along with the full scope of legal equivalents to which such claims are entitled.

Various embodiments of the present subject matter relate to user interfaces, data algorithms and trained models, data training methods and data processing systems, and computing systems and devices used in connection with a closed-loop neurostimulation programming system. As an example, aspects refer to user interface screens and inputs to guide patients during a data collection process that collects (or, causes the collection of) relevant input data for training a programming model—with such relevant input data referred to herein as “training data”. The training data, once collected, can be used to train, retrain, or modify operation of a neurostimulation programming model. User interface screens can obtain data from a human subject (patient) who is undergoing neurostimulation treatment and who can provide feedback on the efficacy or suitability of the neurostimulation treatment. In addition, various outputs may be provided in these user interfaces.

User interface screens, for example, can output suggestions, recommendations, instructions, guidance, or other information to inform the human subject on how to collect more training data, and what programs or programming settings to use to collect adequate amounts and types of training data. Such user interface screens can show a patient their “training progress” on use of different programs or types of programs, and inform the user about what data or type of data has not been collected. As one specific example, a training progress (e.g., a metric or score on a scale of 0% to 100%) may be generated and displayed to a user to indicate how much input has been collected for training a programming model, what types of activities or programming may be used to complete the training of the programming model, and what actions can be taken to begin or to complete model training.

Various forms of data processing may occur based on the input data collected from a human user, or similar data processing may be used to generate the outputs that assist the data collection and model training process. The data processing may produce a patient-specific recommendation of different programs or activities that help identify “gaps” in input data, which can then lead to new data collection on programs or parameter settings that have not yet been used. Such features provide useful techniques to implement and improve a closed-loop programming system, which can automatically select or recommend therapies from new programming of a neurostimulation device. This can lead to a variety of optimizations in the use and deployment of new programs, modification of existing programs, optimization of program usage and schedules, and other neurostimulation device improvements.

As discussed in more detail below, some examples of input data that can be considered for training of a closed-loop programming system includes: patient use of multiple (i.e., different) programs and program parameters; patient answers to questionnaires or patient feedback that provides outcome data (e.g., subjective answers on whether the program was helpful or not); objective metrics or data from the use of the stimulator or programming system (e.g., amount and time of use); the like. It is often difficult to collect such data from patients and determine whether a particular program is responsible for beneficial effects. At times, patients may not provide certain data, or may not provide a diversity of certain types of data. As a further complication, patient goals and objectives may change over time, which may require retraining of a programming model or a modification to how the programming model data is used. As a result, many types of existing programming systems may have incomplete or incorrect information for a particular patient, and programming systems may not be able to provide accurate closed loop model training or programming recommendations. These and other technical challenges are addressed by the techniques discussed below.

The following describes a “data collection platform”, “data processing system”, and “data service” that generally refers to portions of a compute platform (e.g., a combination of hardware, firmware and software) with a set of capabilities for collecting, processing, and generating data in connection with closed-loop neurostimulation programming and the user interfaces discussed below. A compute platform may be a single platform (e.g., at a single cloud service) or may be organized as more than one platform (e.g., among multiple distributed computing locations) that are configured to cooperate with each other. A compute platform may obtain data from one device or from among more than one device. Thus, for example, a therapy device such as an implanted neuromodulation device may provide some portion of the collected data, and a user device (e.g., smartphone) with an interactive user interface (e.g., provided by a smartphone app) may provide another portion of the collected data. A compute platform may also obtain data from other sensor(s) and other data source(s).

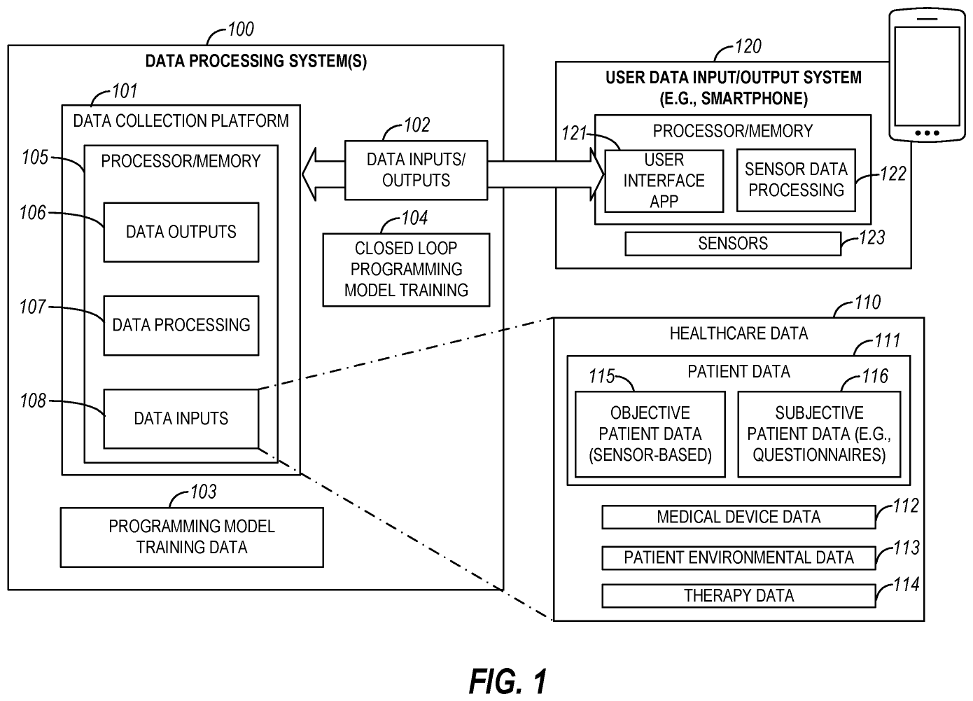

FIG. 1 illustrates, by way of example, an embodiment of a data processing system 100 configured to collect and analyze healthcare-related data in connection with the use of neurostimulation programs and closed-loop neurostimulation programming. The illustrated data processing system 100 is configured to include at least one data collection platform 101, which operates to collect and provide data inputs/outputs 102. The data collection platform 101 is configured to process (e.g., filter, extract, transform) the input data to produce programming model training data 103, which then can be used for one or more iterations of closed loop programming model training 104. The data collection platform 101 can also generate output data in the form of recommendations, suggestions, and other information which is relevant to the collection of input data.

The data processing system 100 may be implemented at one or more server(s) or other systems remotely located from the patient. The data processing system 100 may use various network protocols to communicate and transfer data through one or more networks which may include the Internet. The data processing system 100 and data collection platform 101 may include at least one processor configured to execute instructions stored in memory (e.g., depicted as processor(s)/memory 105) to generate or evaluate data outputs 106, to obtain or evaluate data inputs 108, and to perform data processing 107 on both inputs and outputs and accompanying training data. For instance, the data inputs 108 may be processed to generate the programming model training data 103, which represents a transformed or refined version of data based on actual patient usage and patient feedback (e.g., feedback that indicates what settings have been effective or have not been effective).

The data inputs 108 may include information obtained from the patient directly, and may include various types of healthcare data 110 associated with the patient or the treatment. Examples of healthcare data 110 may include patient data 111, medical device data 112, patient environmental data 113, therapy data 114, or various combinations thereof. The patient data 111 may include objective data 115 such as data collected from physiological sensor(s) and subjective data 116 such as data collected from user-answered question(s) (e.g., “How do you rate your pain?”). Additional examples of how objective and subjective data is collected from a patient is discussed in more examples below.

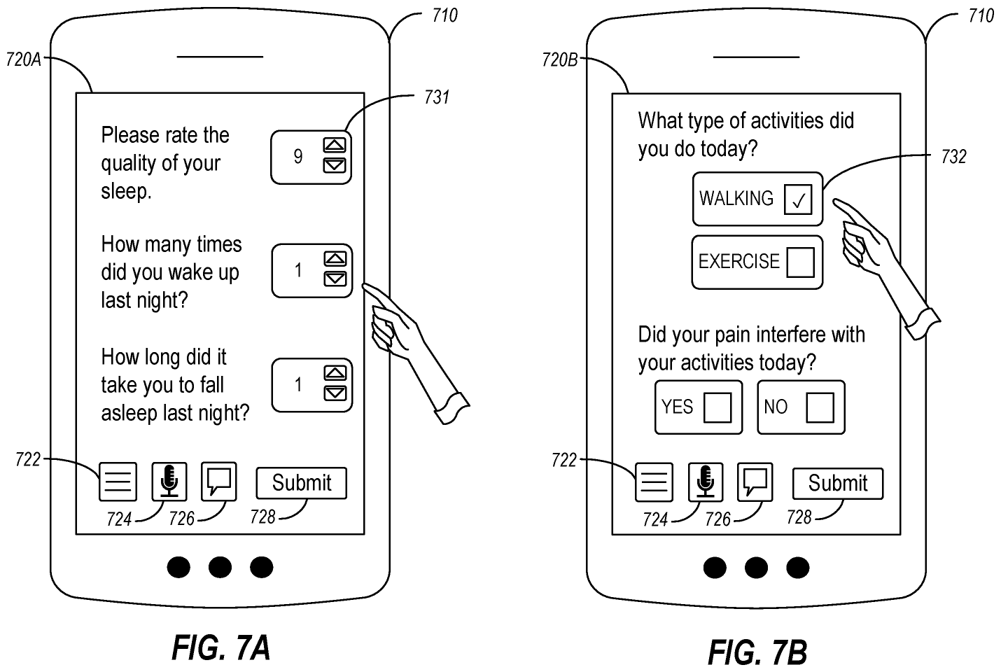

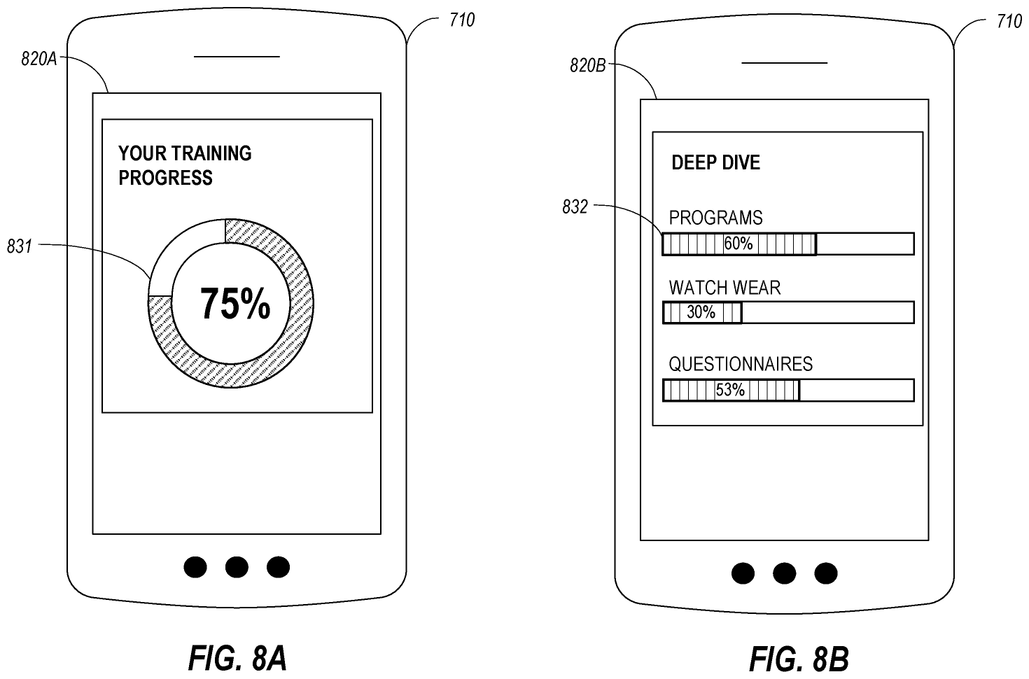

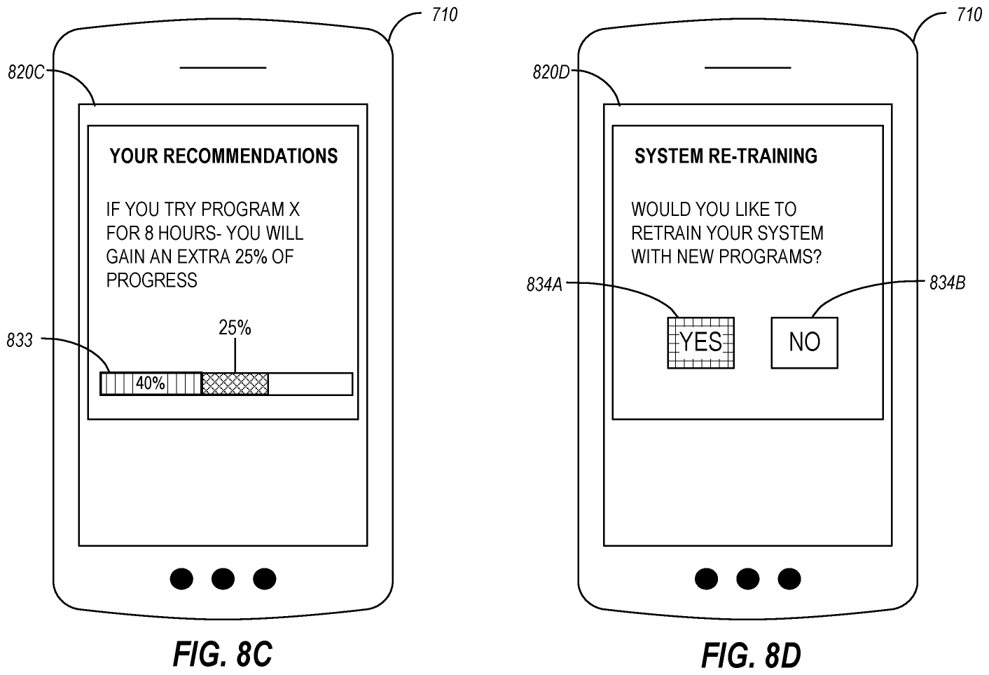

The user data input/output system 120 may be implemented at one or more devices located at or operated by the patient, such as via a smartphone, personal computer, tablet, smart home device, a remote programmer, a programming device, or another compute device or platform capable of collecting input and providing output. The user data input/output system 120 may include at least one processor configured to execute instructions stored in memory (e.g., depicted as processor(s)/memory 105) to provide or generate the data input(s) and outputs 102 for receiving or collecting healthcare data. One such example is a user interface application 121 implemented as a graphical user interface (GUI). Examples of GUIs for data inputs and outputs include smartphone apps that are illustrated in FIGS. 7A to 8D, discussed below. Another example is sensor data processing 122 which can be used to determine the patient physiological state (e.g., amount of activity, duration of sleep) when a particular program is in use.

The user data input/output system 120 may include or interface with sensors 123 such as physiological sensors that can detect patient state information (e.g., activity, sleep), event(s), indicators of device usage, patient compliance with data collection and/or therapy, or various combinations thereof. The user data input/output system 120 may directly or indirectly capture measurements from the sensors 123 or from associated external systems. Examples of external system(s) include remote controls, programmers, phones, tablets, smart watches, personal computers, and the like. The external system may be configured to provide the medical device data 112, the patient environmental data 113, or the therapy data 114 from an associated medical device.

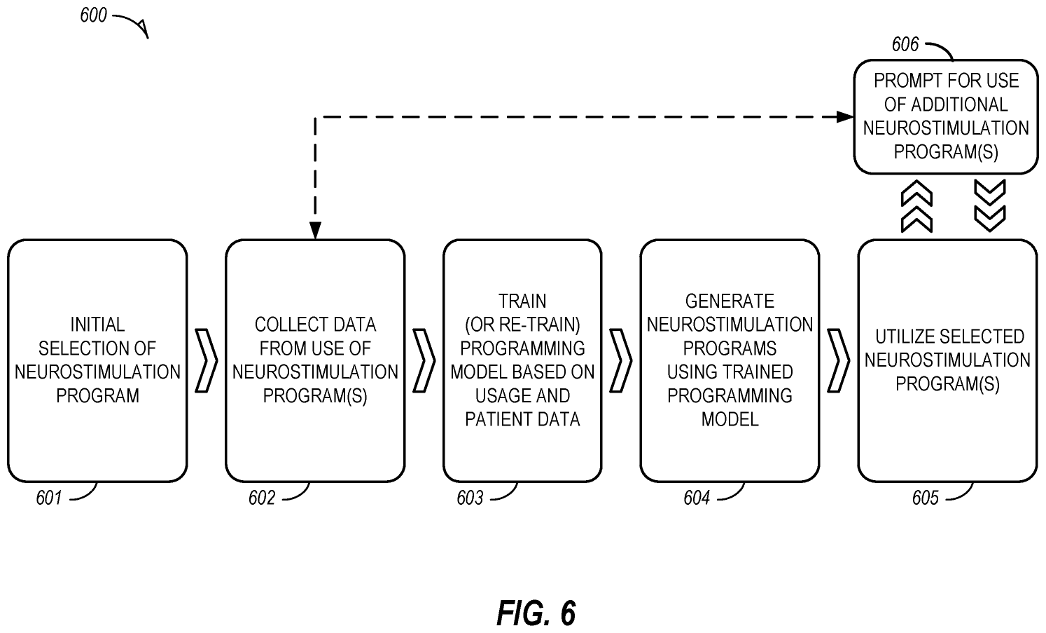

The closed loop programming model training 104 operates to update a programming model by training, re-training, or updating a model (e.g., an artificial intelligence model, such as a neural network) based on the programming model training data 103. One or multiple instances of a model may be trained to generate programs and program parameters, including with one or more patient-specific models. The closed loop programming model training 104 may consider other aspects of patient-specific or population data such as the healthcare data 110, sensor data, and rules and information from a variety of data sources. More details on an example closed loop programming workflow is depicted in FIG. 6, discussed below.

In some examples, the data collection platform 101 and the data processing system 100 may directly interface with one or more medical device(s), external system(s) or other healthcare related data source(s) to collect the healthcare data 110. One or more of the medical device(s), external system(s) or other healthcare-related data source(s) may include technology used by the data processing system 100 to collect data, and thus may form part of the data collection platform 101. Examples of medical devices include implantable and wearable devices. The medical device may be configured to only collect data, to only deliver therapy, or to both collect data and deliver therapy. The medical device may be configured to collect and provide medical device data such as device model, configuration, settings, and the like. Thus, the medical device may provide the patient data 111, the medical device data 112, the environmental data 113, and therapy data 114, particularly in specialized neurostimulation programming environments.

Other healthcare-related data source(s) may include patient data received via a provider's server that stores patient health records. For example, patients may use a patient portal to access their health records such as test results, doctor notes, prescriptions, and the like. Other healthcare-related data sources may include apps on a patient's smartphone or other computing device, or the data on a server accessed by those apps. By way of example and not limitation, this type of data may include heart rate, blood pressure, weight, and the like collected by the patient in their home. In another example, an app on a phone or patient's device may include or may be configured to access environmental data such as weather data and air quality information or location elevation data such as may be determined using cellular networks and/or a global positioning system (GPS). Weather data may include, but is not limited to, barometric pressure, temperature, sunny or cloud cover, wind speed, and the like.

The data inputs/outputs 102 may be provided with data transferred via at least one network. The data transfer may use various network protocols to communicate and transfer data through one or more networks which may but does not necessarily include the Internet and/or various wireless networks, which may include short range wireless technology such as Bluetooth. The data may be transferred directly from at least one of the external systems and/or may be transferred directly from at least one of the medical device(s). Further, the external system(s) may be configured to receive data from an associated medical device(s) and/or receive data from other healthcare-related data source(s), and then transfer the data through the network(s) to the data receiving system(s).

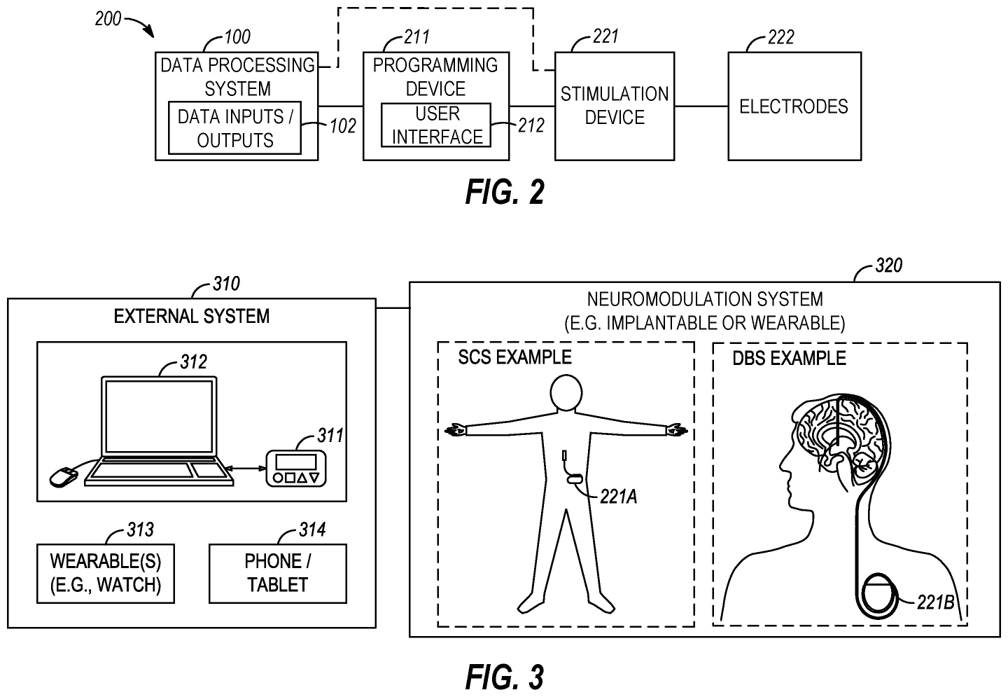

FIG. 2 illustrates a neuromodulation system as an example of a medical device system. The medical device may be configured, by way of example and not limitation, to deliver an electrical therapy using one or more electrodes 222 provided by a stimulation device 222. In the illustrated embodiment, the medical device may be a neuro stimulation device, and the system may be a neuromodulation system 200.

The illustrated neuromodulation system 200 includes electrodes 222, the stimulation device 221 and a programming system such as a programming device 211. The programming system may include multiple devices. The electrodes 222 are configured to be placed on or near one or more neural targets in a patient. The stimulation device 221 is configured to be electrically connected to the electrodes 222 and deliver neuromodulation energy, such as in the form of electrical pulses, to the one or more neural targets though the electrodes 222. The system may also include sensing circuitry to sense a physiological signal, which may but does not necessarily form a part of stimulation device 221. The delivery of the neuromodulation is controlled using a plurality of modulation parameters that may specify the electrical waveform (e.g., pulses or pulse patterns or other waveform shapes) and a selection of electrodes through which the electrical waveform is delivered. In various embodiments, at least some parameters of the plurality of modulation parameters are programmable by a user, such as a physician or other caregiver, using one or more program. The programming device 211 thus provides the user with accessibility to the user-programmable parameters. The programming device 211 may also provide the use with data indicative of the sensed physiological signal or feature(s) of the sensed physiological signal.

In various embodiments, the programming device 211 is configured to be communicatively coupled to the stimulation device 221 via a wired or wireless link. In various embodiments, the programming device 211 includes a user interface 212 such as a graphical user interface (GUI) that allows the user to set and/or adjust values of the user-programmable modulation parameters. The user interface 212 may also allow the user to view the data indicative of the sensed physiological signal or feature(s) of the sensed physiological signal and may allow the user to interact with that data. The data is provided to a data processing system 100 which provides various data inputs and outputs 102 to assist the user with the operation, training, configuration, maintenance, or improvement of the stimulation device 221, such as for the collection of training data as discussed herein.

In some examples, the stimulation device 221 and the programming device 211 may contribute to the training data provided to the data processing system 100. The user interface 212 of the programming device 211 may be used to allow the user to answer questions to provide healthcare-related data, although other devices (such as a patient smartphone) may be used as discussed below. Therapy parameters, programming selection, electrode selection, and other operational parameters may also provide healthcare-related data for use by the data processing system 100. Additional sensor(s) may also provide healthcare-related data for use by the data processing system.

FIG. 3 illustrates, by way of example and not limitation, the neurostimulation system of FIG. 2 implemented in a spinal cord stimulation (SCS) system or a deep brain stimulation (DBS) system. The illustrated neuromodulation system 320 connects with an external system 310 that may include at least one programming device. The illustrated external system 310 may include a clinician programmer 312 configured for use by a clinician to communicate with and program the neurostimulator, and a remote control device 311 configured for use by the patient to communicate with and program the neurostimulator. For example, the remote control device 311 may allow the patient to turn a therapy on and off and/or may allow the patient to adjust patient-programmable parameter(s) of the plurality of modulation parameters (e.g., by switching programs).

FIG. 3 further illustrates the neuromodulation system 320 as an ambulatory medical device, such as implemented by stimulation device 221A or stimulation device 221B. Examples of ambulatory devices include wearable or implantable neuromodulators. The external system 310 may include a network of computers, including computer(s) remotely located from the ambulatory medical device that are capable of communicating via one or more communication networks with the programmer 312 and/or the remote control device 311. The remotely located computer(s) and the ambulatory medical device may be configured to communicate with each other via another external device such as the programmer 312 or the remote control device 311.

The external system 310 may also include one or more wearables 313 and a portable device 314 such as a smartphone or tablet. In some examples, the wearables 313 and the portable device 314 may allow a user to obtain and provide input data, including to answer questions (e.g., on a phone/tablet screen) or to input sensor data values (e.g., from a physiologic sensor of a wearable) as part of the data collection process. In some examples, the remote control device 311 and/or the programmer 312 also may allow a user (e.g., patient and/or clinician or rep) to answer questions and provide input as part of the data collection process. The remote control device 311 and/or the programmer 312 may be used to provide other aspects of the input data, including usage data of various neurostimulation programs, events associated with such programs, and the like.

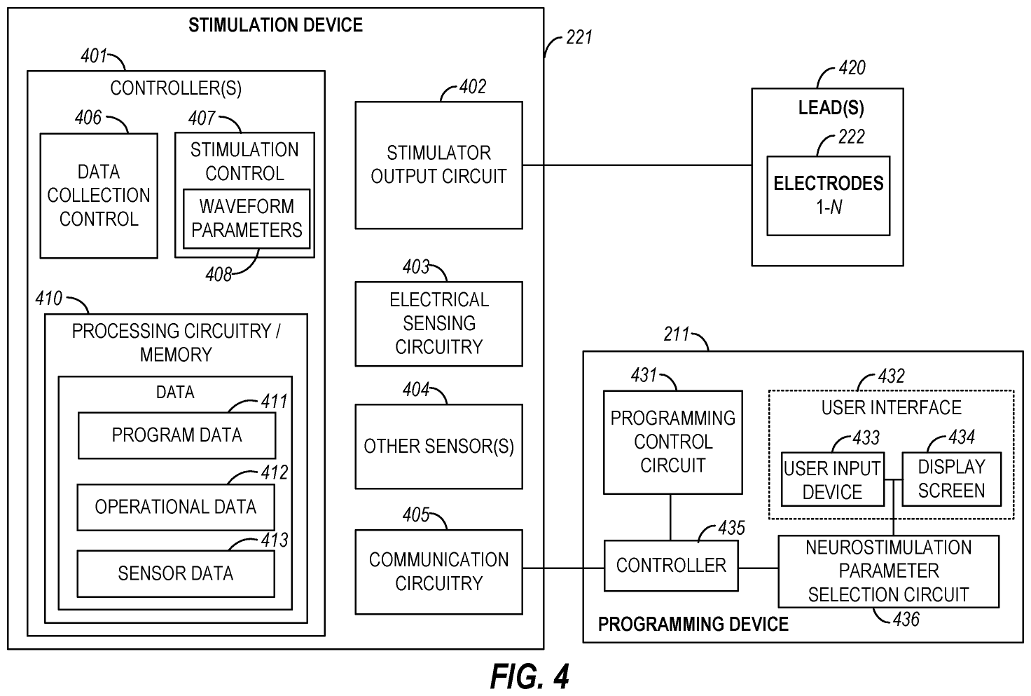

FIG. 4 illustrates, by way of example, an embodiment of a neuromodulation device, such as may be implemented as the stimulation device 221 illustrated in FIGS. 2 and 3. The stimulation device 221 may be configured to be connected to electrode(s) 222, illustrated as N electrodes, via one or more leads 420. Any one or more of the electrodes 222 may be configured for use to deliver modulation energy, sense electrical activity, or both deliver modulation energy and sense electrical activity. The stimulation device 221 may include a stimulator output circuit 402 configured to deliver modulation energy to electrode(s). The stimulator output circuit 402 may be configured with multiple (e.g., two or more) channels for delivering modulation energy, where each channel may be independently controlled with respect to other channel(s). For example, the stimulator output circuit 402 may have independent sources such as independent current sources or independent voltage sources.

In various examples, the electrodes 222 may include a stimulation electrode or a sensing electrode. The stimulation electrode is configured for use in delivering modulation energy, and the sensing electrode is configured for use in sensing electrical activity. As illustrated, the stimulation electrode may also be used in sensing electrical activity, and the sensing electrode may also be used in delivering modulation energy. Thus, the term “stimulation electrode” does not necessary exclude the electrode from also being used to sense electrical activity; and the term “sensing electrode” does not necessarily exclude the electrode from also being used to deliver modulation energy.

The stimulation device 221 may include electrical sensing circuitry 403 configured to receive sensed electrical energy from the electrode(s), such as may be used to sense electrical activity in neural tissue or muscle tissue. The sensing circuitry may be configured to process signals in multiple (e.g., two or more) channels. By way of example and not limitation, the electrical sensing circuitry 403 may be configured to amplify and filter the signal(s) in the channel(s).

The controller 401 may be configured to detect one or more features in the sensed signals. Examples of features that may be detected include peaks (e.g., minimum and/or maximum peaks including local peaks/inflections), range between minimum/maximum peaks, local minima and/or local maxima, area under the curve (AUC), curve length between points in the curve, oscillation frequency, rate of decay after a peak, a difference between features, and a feature change with respect to a baseline. Detected feature(s) may be fed into a control algorithm, which may use relationship(s) between the feature(s) and waveform parameter(s) to determine feedback for closed-loop control of the therapy. Some embodiments of the stimulation device 221 may include or be configured to receive data from other sensor(s) 404. The other sensor(s) 404 may include physiological sensor(s), environmental sensor(s), or proximity sensor(s).

The stimulation device 221 may include a controller 401 operably connected to the stimulator output circuit 402 and the electrical sensing circuitry 403. The controller 401 may include a stimulation control 407 (e.g., logic) configured for controlling the stimulator output circuit 402. For example, the stimulation control 407 may include start/stop information for the stimulation and/or may include relative timing information between stimulation channels. The stimulation control 407 may include waveform parameters 408 (e.g., associated with a program or a defined set of parameters) that control the waveform characteristics of the waveform produced by the stimulator output circuit 402. The waveform parameters 408 may include, by way of example and not limitation, amplitude, frequency, and pulse width parameters. The waveform parameters 408 may include, by way of example and not limitation, regular patterns such as patterns regularly repeat with same pulse-to-pulse interval and/or irregular patterns of pulses such as patterns with variable pulse-to-pulse intervals. The waveform parameters may, but do not necessary, define more than one waveform shape (e.g., including a shape other than square pulses with different widths or amplitudes). The stimulation control 407 may be configured to change waveform parameter(s) (e.g., one or more waveform parameters) in response to user input and/or automatically in response to feedback.

The controller 401 may include a data collection control 406 configured for use by the stimulation device 221, and the data collection platform 101 of a data processing system 100 (see FIGS. 1-2), to collect healthcare related data. The controller 401 may include a processor and/or memory 410 (e.g., with instructions) for use to control the data collection using the data collection control 406 and control the stimulation via the stimulation control 407. The memory may also provide storage for storing different types of collected healthcare-related data, such as program data 411, operational data 412, sensor data 413, and the like. Examples of sensor data 413 collected by the stimulation device 221 or other devices discussed herein may include, by way of example and not limitation, heart rate, heart rate variability, oxygen saturation, activity, posture, steps, gait, temperature, evoked compound action potentials (ECAPS), electromyograms (EMGs), electroencephalograms (EEGs), weight, blood pressure, and the like. Examples of program data 411 may include, by way of example and not limitation, stimulation settings such as amplitude, pulse width, pulse frequency period, duration of burst of pulses, active electrodes, electrode fractionalization controlling the distribution of energy (e.g., current) to active electrodes, waveforms, pulse patterns including various complex patterns, and the like. Examples of operational data 412 of the stimulation device 221 may include, by way of example and not limitation, electrode-tissue impedance, fault conditions, battery information such as battery health, battery life, voltage, charge state, charging history if rechargeable, MRI status, Bluetooth connection logs, connection with Clinician Programmer, hours of operation/duration of implant, and the like. Other device information may include device model and lead model.

The neuromodulation device may include communication circuitry 405 configured for use to communicate with other device(s) such as a programming device 211, remote control, phone, tablet and the like. The healthcare-related data may be transferred out from the neuromodulation device for transfer to a data processing system, as discussed above. As shown, a programming device 211 includes a programming control circuit 431, a user interface 432 (e.g., including a user input device 433 such as a buttons and a display screen 434), a controller 435, and other components (e.g., an external communication device, not shown) to effect programming of a connected neurostimulation device. The operation of the neurostimulation parameter selection circuit 436 enables selection, modification, and implementation of a particular set of parameters or settings for neurostimulation programming (e.g., via selection of a program, specification by a closed-loop or open-loop programming process, specification by a patient or clinician, or the like).

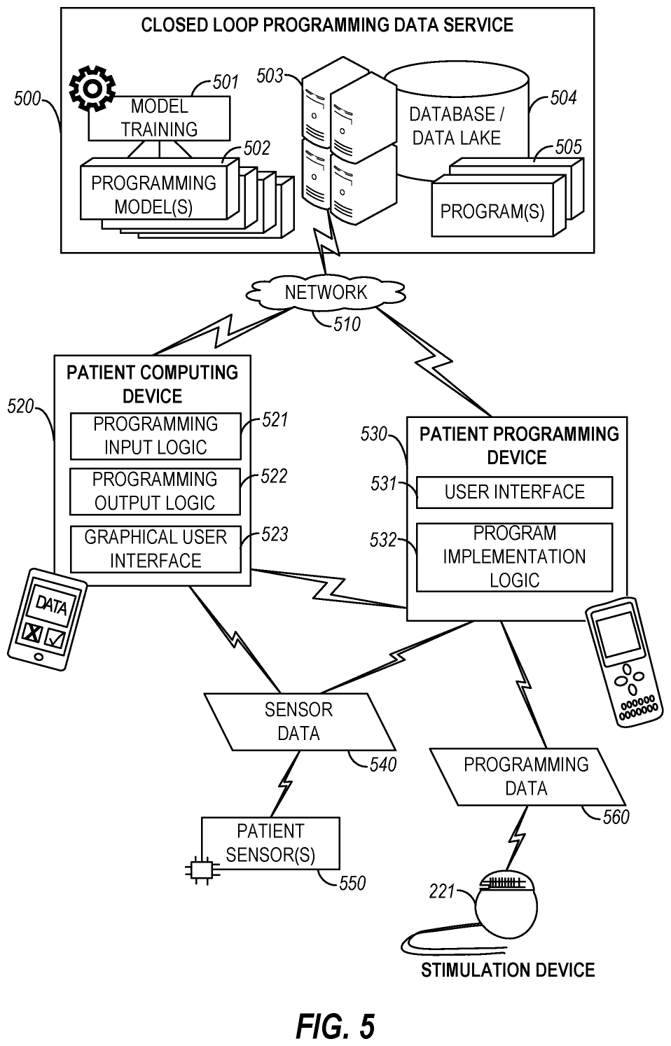

FIG. 5 illustrates, by way of example, an embodiment of data interactions among a data processing system implemented as a closed loop programming data service 500, for operation of a stimulation device 221 with use of closed loop programming. At a high level, a closed loop programming data service 500 uses a trained model (one of programming models 502) to generate programming settings and parameters (e.g., in one or more programs 505) that are customized to the human patient. The closed loop programming data service 500 includes compute hardware 503 to control the model training 501 and other data processing operations, such as to generate or control diagnostic actions, alerts, programming recommendations, or programming actions. The programming settings and parameters may be implemented automatically or manually on the stimulation device 221 (e.g., using the programming techniques referenced above) or via a patient computing device 520 or patient programming device 530.

The closed loop programming data service 500 communicates with one or both of the patient computing and programming devices 520, 530, to obtain training data for use by model training logic 501. The training data may be stored in a database 504 or another large-scale data store (e.g. data lake) for the patient or a population of patients. The closed loop programming data service 500 may also include data analysis or processing engines (not shown) that parse and determine a state of a particular patient from various inputs and correlated program usage (e.g., to determine what programs and programming settings are beneficial or not beneficial to the patient). In some examples, the state of treatment may be based on correlating the historical use of a neurostimulation program or set of parameters with the current state of a patient (e.g., identifying that a pain condition became worse or better after beginning use of a particular program).

The closed loop programming data service 500 may also analyze a variety of forms of patient input and patient data related to usage of a neurostimulation program or neurostimulation programming parameters. For instance, the closed loop programming data service 500 may receive information from program usage, questionnaire selections, or even text input originating from a human patient, via the patient computing and programming devices 520, 530. In addition to providing recommended programs, the closed loop programming data service 500 may also provide therapy content and usage recommendations to the patient computing and programming devices 520, 530.

A patient may provide training data (input data) via the patient computing device 520 or the patient programming device 530. Additional detail of how input data is collected is discussed with reference to the data processing logic and user interfaces discussed in more detail below. In an example, the patient computing device 520 is a computing device (e.g., personal computer, tablet, smartphone) or other form of user-interactive device which receives and provides interaction with a patient using a graphical user interface 523, with use of programming input logic 521 and programming output logic 522. For instance, the programming input logic 521 may receive input from a patient via questionnaires, surveys, messages, or other inputs. The inputs may provide text related to pain or satisfaction, which can be used to identify a psychological or physiological state of the patient, neurostimulation treatment results, or related conditions.

A patient programming device 530 is depicted as including a user interface 531 and program implementation logic 532. The program implementation logic 532 specifically may provide the patient with the ability to implement or switch to particular programs generated by closed loop programming data service 500. Other forms of closed-loop programming may also include the receipt of instructions, recommendations, or feedback (including clinician recommendations, behavioral modifications, etc., selected for the patient) that are automatically selected based on detected conditions.

The closed loop programming data service 500 may also utilize sensor data 540 from one or more patient sensors 550 (e.g., wearables, sleep trackers, motion tracker, implantable devices, etc.) among one or more internal or external devices. The sensor data 540 may be used to determine a customized and current state of the patient condition or neurostimulation treatment results. In various examples, the stimulation device 221 also includes sensors which contribute to the sensor data 540 to be evaluated by the closed loop programming data service 500.

In an example, the patient sensors 550 are physiological or biopsychosocial sensors that collect data relevant to physical, biopsychosocial (e.g., stress and/or mood biomarkers), or physiological factors relevant to a state of the patient. Examples of such sensors might include a sleep sensor to sense the patient's sleep state (e.g., for detecting lack of sleep), a respiration sensor to measure patient breathing rate or capacity, a movement sensor to identify an amount or type of movement, a heart rate sensor to sense the patient's heart rate, a blood pressure sensor to sense the patient's blood pressure, an electrodermal activity (EDA) sensor to sense the patient's EDA (e.g., galvanic skin response), a facial recognition sensor to sense the patient's facial expression, a voice sensor (e.g., microphone) to sense the patient's voice, and/or an electrochemical sensor to sense stress biomarkers from the patient's body fluids (e.g., enzymes and/or ions, such as lactate or cortisol from saliva or sweat). Other types or form factors of sensor devices may also be utilized.

FIG. 6 depicts a workflow 600 for closed-loop programming of a neuromodulation system, such as the stimulation device 221 discussed above. It will be understood that additional user or automated actions, commands, and data operations may be involved as part of closed-loop programming, and are omitted for purposes of simplicity.

At 601, the workflow begins with an initial selection or implementation of a particular neurostimulation program (or, multiple neurostimulation programs). This initial selection or implementation may be provided from clinician programming, or patient selection of the program(s).

At 602, training data, including subjective and objective data related to the use of neurostimulation program(s), is collected from use of the neurostimulation program(s). This training data is stored (e.g., in a database or in a data lake 504).

At 603, a neurostimulation programming model (e.g., one or more of models 502) is trained or re-trained, based on the training data. This training data includes information from program usage and patient data, and may include various forms of objective and subjective data.

At 604, one or more neurostimulation programs (e.g., one or more of programs 505) are generated using the trained (or, re-trained) programming model. The neurostimulation programming model in some examples may be a patient-specific model, although the model may also be trained from or specific to population data or data from multiple patients having a particular medical condition.

At 605, the one or more neurostimulation programs (e.g., new programs, modified programs) are deployed to the stimulation device. As these program(s) are made available, the programs may be selected by the patient and used for patient treatment.

At 606, the patient is prompted to provide inputs and to use additional (e.g., new, or revised) neurostimulation programs or program settings. The training data from the use of the additional neurostimulation programs is again collected at 602, and used for additional training operations, to repeat operations 603 to 606.

The next examples include types of user interfaces and interactions that provide questionnaires and recommendations to a human patient. It will be understood that the data processing performed by the present approaches may occur on a variety of user-interactive interfaces, including text, graphical, or voice inputs and outputs. Although a smartphone app is depicted, other forms of multi-modal input via multiple channels (e.g., SMS text messages, an email, a website, a chatbot, a metaverse session, etc.) may also be used.

FIGS. 7A to 7B depict data input user interfaces 720A, 720B (e.g., a smartphone app graphical user interface) which provide output on a user device 710 (e.g., a smartphone). In various examples, a patient's phone, computer, tablet, or programming remote control (or, multiple of these devices) may provide the illustrated user interfaces.

FIG. 7A depicts the data input user interface 720A as including a number of patient inputs regarding sleep, including a selection 731 to provide a subjective answer on the quality of sleep (e.g., a numerical answer on a scale of 1 to 10, with 10 being the best quality). FIG. 7B similarly depicts the data input user interface 720B as including a number of patient inputs regarding activity, including a selection 732 of one or more different activities, as well as a binary “YES” or “NO” button selection for a question regarding the activities. In either example, the patient answers may be received using other techniques. The user interface may also provide guidance, warnings, validations, or informative information to help gather the input from the user.

The questions may be provided in connection with a time-based questionnaire (e.g., a morning questionnaire, an evening questionnaire) that includes a fixed number of prompts (e.g., a series of questions). A questionnaire may be used to determine a particular patient state at one or more times (e.g., a first patient state at the time that a first program was in use, a second patient state at the time that a second program was in use, etc.).

The user interfaces 720A, 720B are also depicted to include other user input functionality, such as menu selection functionality 722, voice input functionality 724, text input functionality 726, and a submit control functionality 728. A variety of dynamic user interface features may also be used based on validating or entering user data. In other examples, input data may be provided from non-graphical or non-text inputs, such as voice interactions controlled by a chatbot or by virtual assistants or agents (e.g., Amazon® Alexa, Google® Assistant, Apple® Siri, etc.

FIGS. 8A to 8D depict output data user interfaces 820A, 820B, 820C, 820D, providing various aspects of informative data, such as within an app executing on the smartphone mobile device 710. Additional menus and user interface functionality are not shown for purposes of simplicity.

FIG. 8A depicts the user interface 820 as including a graphical representation 831 of training progress. Specifically, this screen depicts a graphic to represent a metric of training progress (e.g., on a scale from 0-100%) to indicate how much input has been given to training the model (e.g., how much training data has been collected). As an example, the graphical representation 831 may show 0% when the patient first begins usage of the neurostimulation program, and has not collected any training data or performed any training activities. The graphical representation 831 may show 100% when a sufficient amount of training data has been collected, and personalized programming/closed-loop optimization can begin. However, in other examples, the metric may represent other values or milestones.

In specific examples, the metric may relate to an amount of usage of a particular neurostimulation program, and the collection of input data (e.g., patient surveys, questionnaires, and wearable sensor data) that corresponds to the usage of the particular neurostimulation program. In other examples, the metric may relate to the amount of usage of multiple neurostimulation programs. For instance, if a patient uses one neurostimulation program with the same parameters for a long time, it may be unlikely that training data will progress after the initial information gain. However, if a patient uses a variety of programs with many different outcomes, progress can move more rapidly.

FIG. 8B depicts the user interface 820B as including a graphical representation 832 on multiple measurements of training data. Specifically, the graphical representation 832 provides a score on multiple measurements of training data sources. For instance, this may include a first measurement representing an amount of data collected from the usage of multiple neurostimulation programs; a second measurement representing an amount of data collected from the use of wearable devices (e.g., sensor data from a smartwatch during use of the programs); and a third measurement representing an amount of data collected from inputs in user questionnaires. Thus, the user interface 820B can enable a patient to not only see an overall “score” for training, but also to identify where “input gaps” are in the data, and what areas that the user can improve.

FIG. 8C depicts the user interface 820C as including a graphical representation 833 with recommendations of how to increase the collection of training data. Here, this graphical representation 833 shows the amount of training data collected to date (e.g., 40% of total training data required to start training), and the amount of training data that can be collected from the recommendation (e.g., 25% more data that can be collected if the recommendation is followed). In further examples, various aspects of gamification (e.g., bonuses, deadlines, reminders, etc.) can be used to encourage a user to perform certain data collection activities (e.g., use different programs, apply different settings, etc.).

FIG. 8D depicts the user interface 820D as including graphical selection options 834A, 834B, which can trigger or cause programming model retraining. For instance, these graphical selection options 834A, 834B may be presented once a threshold (minimum) amount of training data has been collected at a user device. The selection of a retraining option can be accompanied by the transfer of data to a remote system (e.g., the closed loop programming data service 500). Other user interaction actions can include options to restart training; to modify or add training for additional therapies; or to refocus training on different goals.

Although the preceding examples refer to the use of individual programs, it will be understood that training data may also be collected from the use of customized programming settings (e.g., changes in amplitude, intensity, waveforms, etc.) that are controlled by a user. Further, training data may be collected after providing recommendations for specific activities (e.g., program changes, programming setting changes, personal activities, etc.) that are delivered via one or more schedules, tasks, and goals.

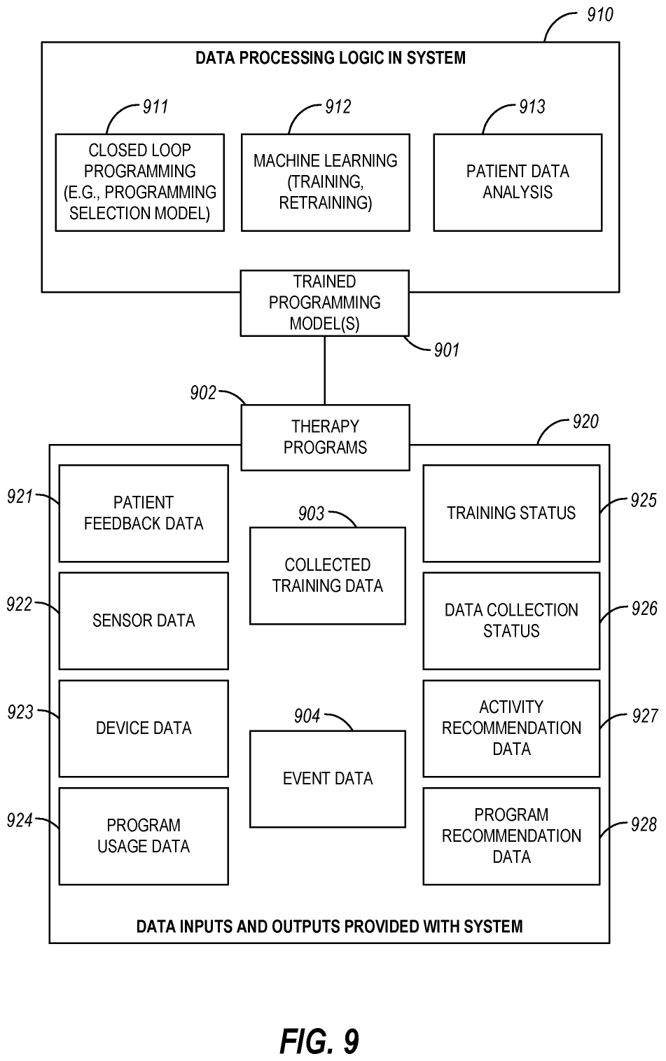

FIG. 9 illustrates data types and processing logic associated with the data input or output examples for closed-loop programming, as discussed above. The data processing logic 910 (e.g., implemented by data processing system 100, programming data service 500, or other systems discussed above) may operate to produce one or more trained programming models 901, such as programming models that generate neurostimulation programming parameters (e.g., programming selection models, or programming generation models).

In an example, the data processing logic 910 provides implementations of closed-loop programming logic 911, such as logic that produces a program selection model or program generation model for closed-loop programming of a neurostimulation device. The data processing logic 910 may also include machine learning logic 912 that performs training or retraining of the program selection model or program generation model. The data processing logic 910 may also include patient data analysis logic 913, used to analyze a number of relevant patient data values (e.g., from patient input, healthcare data, etc.).

The trained programming models 901 are used to produce one or more therapy programs 902. The usage of the therapy programs 902 is associated with multiple data inputs and outputs 920 provided with (or from) the system. As non-limiting examples, such data inputs may include objective and subjective data provided in: patient feedback data 921; sensor data 922; device data 923; program usage data 924. As non-limiting examples, such data outputs may include training status 925; data collection status 926; activity recommendation data 927; program recommendation data 928.

Other types of objective data and subjective data may be collected or output in the data inputs and outputs 920. Objective data, as used herein, is data that can obtained from a measurement or direct observation. Objective data may be measured by a sensor and may be provided via user input when the user has access to objectively determined information. Categories of objective data may include physiological parameter data, therapy data, device data, and environmental data. By way of example and not limitation, physical parameter data may include data such as: heart rate, blood pressure, respiration rate, activity, posture, electromyograms (EMGs), neural responses such as evoked compound action potentials (ecaps), glucose measurements, oxygen levels body temperature, oxygen saturation and gait. By way of example and not limitation, therapy data may include: neuromodulation programs, therapy on/off schedule, dosing, neuromodulation parameters such as waveform, frequency, amplitude, pulse width, period, therapy usage and therapy type. By way of example and not limitation, device data may include: battery information (voltage, charge state, charging history if rechargeable), impedance data, faults, device model, lead models, MRI status, Bluetooth connection logs, connection history with Clinician's Programmer (CP). By way of example and not limitation, environmental data may include: temperature, air quality, pressure, location, altitude, sunny, cloudy, precipitation, etc. Subjective data can include information received from the patient or another human user (e.g., caregiver, clinician, etc.). For example, the patient's quantification of pain can provide subjective data. Subjective data may generally involve user-inputted data. Examples of subjective data include questions with free text answers, multiple choice questions, question tree(s), and different question subject(s). Other data may be stored and/or transferred, including detected event data 904 to track events (e.g., that trigger a response, change data resolution), contextual data, time data, and the like. The event(s), context(s) and time may be detected by the system or may be provided via user input.

The collected training data 903 may be communicated at regular intervals, or on-demand when re-training is requested. The collected training data 903 may be pre-processed at a medical device or an external device (e.g., phone, tablet, remote control, programmer) by the patient, or at a remote data receiving system. The collected training data 903 may provide an input to the patient data analysis 913 which, by way of example and not limitation, uses the acquired data to identify therapy outcomes or a patient state. After processing, the machine learning logic 912 may operate on the collected training data 903 to develop or refine the closed loop programming 911 and the trained programming model(s) 901. The data processing logic 910, in some examples, may use data imputation, which refers to a process of replacing missing data with substituted values such as may be used to prevent missing data from introducing bias into the model(s) or machine learning.

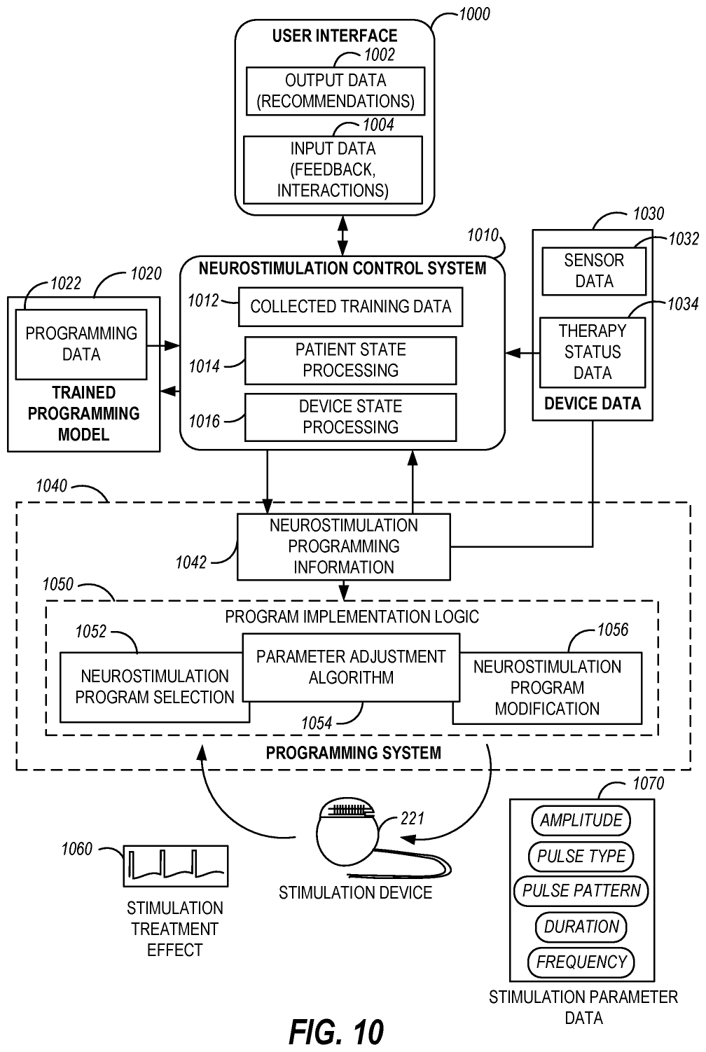

FIG. 10 illustrates, by way of example, an embodiment of a data processing flow for updating the neurostimulation treatment of a human patient, which may occur based on the closed-loop programming training operations discussed above. Specifically, this data processing flow shows how a neurostimulation control system 1010 may perform patient state processing 1014 and device state processing 1016 functions, based on the collected training data 1012 (discussed above) that determine improvements to neurostimulation programming models (e.g., to produce a newly trained programming model 1020, which in turn produces updated programming data 1022 for deployment with new neurostimulation programming information 1042).

In this example, the user interface 1000 provides output data 1002 (e.g., recommendations) to encourage the usage of programs and to help collect data on the usage of neurostimulation programs. The user interface 1000 collects input data 1004 (e.g., survey answers, freeform text, other user interactions), as discussed above, to determine the efficacy or benefit from use of the one or more neurostimulation programs. Such operations and functionality may be consistent with those user interfaces discussed above, although other data inputs and outputs may be used.

FIG. 10 also depicts the evaluation of device data 1030, such as sensor data 1032, therapy status data 1034, and other treatment aspects which may be obtained or derived from the stimulation device 221 or related neurostimulation programming and device operation. The device data 1030 and the inputs received with the user interface 1000 allow a set of training data to be collected, which can be further evaluated with patient state processing functions 1014 and device state processing functions 1016. The trained programming model 1020 generates new or updated programming data 1022 (e.g., programs, program parameter values) based on training performed from the collected training data 1012, and from results of the patient state processing functions 1014 and device state processing functions 1016.

The remainder of the data processing flow illustrates how the neurostimulation control system 1010 implements programming, such as in a closed loop (or partially-closed-loop) system. A programming system 1040 uses programing information 1042 provided from the neurostimulation control system 1010 as an input to program implementation logic 1050. The program implementation logic 1050 may be implemented by a parameter adjustment algorithm 1054, which affects a neurostimulation program selection 1052 or a neurostimulation program modification 1056. For instance, some parameter changes may be implemented by a simple modification to a program operation; other parameter changes may require a new program to be deployed. The results of the parameter or program changes or selection provides various stimulation parameters 1070 to the stimulation device 221, causing a different or new stimulation treatment effect 1060.

By way of example, operational parameters of the stimulation device which may be generated, identified, or evaluated by the neurostimulation control system 1010 may include amplitude, frequency, duration, pulse width, pulse type, patterns of neurostimulation pulses, waveforms in the patterns of pulses, and like settings with respect to the intensity, type, and location of neurostimulator output on individual or a plurality of respective leads. The neurostimulator may use current or voltage sources to provide the neurostimulator output, and apply any number of control techniques to modify the electrical simulation applied to anatomical sites or systems related to pain or analgesic effect. In various embodiments, a neurostimulator program may be defined or updated to indicate parameters that define spatial, temporal, and informational characteristics for the delivery of modulated energy, including the definitions or parameters of pulses of modulated energy, waveforms of pulses, pulse blocks each including a burst of pulses, pulse trains each including a sequence of pulse blocks, train groups each including a sequence of pulse trains, and programs of such definitions or parameters, each including one or more train groups scheduled for delivery. Characteristics of the waveform that are defined in the program may include, but are not limited to the following: amplitude, pulse width, frequency, total charge injected per unit time, cycling (e.g., on/off time), pulse shape, number of phases, phase order, interphase time, charge balance, ramping, as well as spatial variance (e.g., electrode configuration changes over time). It will be understood that based on the many characteristics of the waveform itself, a program may have many parameter setting combinations that would be potentially available for use.

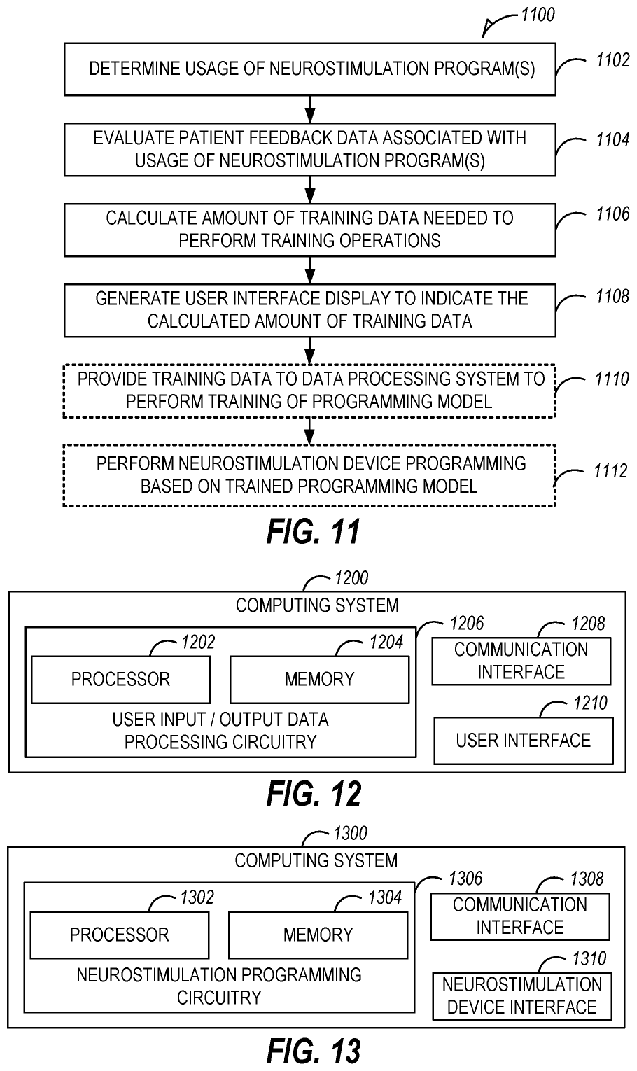

FIG. 11 illustrates, by way of example, an embodiment of a processing method 1100 implemented by a system or device for use to analyze data for neurostimulation programming, e.g., in connection with training data collected for improvement of a programming model used in a closed-loop neurostimulation treatment. For example, the processing method 1100 can be embodied by electronic operations performed by one or more computing systems or devices (including those at a network-accessible remote service) that are specially programmed to implement data analysis and/or neurostimulation data processing operations. In specific examples, the operations of the method 1100 may be implemented through the systems and data flows depicted above, at a single entity or at multiple locations.

In an example, the method 1100 begins at 1102 by determining usage of one or more neurostimulation treatments, such as tracking and evaluating the historical usage of treatments provided by programs. In an example, the particular neurostimulation program treatment is associated with a set of parameters used by a neurostimulation device to deliver neurostimulation to a human patient. For instance, the usage of a particular program or the usage of a particular program having a particular set of program parameters may be tracked, such as to determine usage of the particular program or a set of particular program parameters that define program amplitude, pulse width, rate, cycling on or off times, or the like.

The method 1100 continues at 1104 by evaluating patient feedback data associated with the usage of the neurostimulation program(s). In an example, the patient feedback data is obtained directly (or indirectly) from the human patient, using a user interface or user input mechanisms. Such patient feedback data may be obtained from the human patient using one or more questionnaires, surveys, text entries, or voice inputs. In further examples, the patient feedback data may be obtained or supplemented by data derived from devices that the patient uses, wears, or interacts with. For instance, the method 1100 may include evaluating device data associated with the usage of a particular neurostimulation program that is used by a neurostimulation device to provide the particular neurostimulation treatment, with such device data being obtained from a wearable device or from the neurostimulation device.

In specific examples, the patient feedback data (or, device data) relates to one or more characteristics of: sleep (e.g., sleep quantity, sleep quality, sleep disturbance), pain, movement, fatigue or restfulness, alertness, emotional state, medication state, mobility, or activity, in connection with (during, before, after) use of the particular neurostimulation treatment, including to inquire about the patient state after a program is used or before a program is used. For instance, relevant properties that may be indicated by these characteristics may include: activities of daily living, pain interference, fatigue/restfulness, mood, alertness, medication use (prescribed opioids, prescribed pain meds, sleep medication, over the counter pain medications), effective mobility, mobility minutes, activity minutes, neurostimulation program usage, neurostimulation charging data, neurostimulation amplitude usage, neurostimulation pulse width usage, or neurostimulation pulse rate usage, or combinations or derivatives.

The method 1100 continues at 1106 by calculating an amount of training data needed to perform training operations. In an example, the calculation is performed based on the patient feedback data (and optionally, based on the device data), and the training data is provided at least in part from the patient feedback data (and optionally, from the device data). Such a calculated amount of training data required to perform training operations may be used to identify an availability for retraining of a programming selection model (and, to trigger a user interface display that includes a user-selectable option to cause the retraining of the programming selection model).

The method 1100 continues at 1108 by generating a user interface display to indicate the calculated amount of training data. In an example, the user interface display is generated to include one or more metrics that correspond to the amount of training data required to perform the training operations on a programming selection model. In further examples, the user interface display further includes one or more metrics that corresponds to an amount of training data available to perform the training operations on the programming selection model. Also in further examples, the user interface display further includes one or more values that correspond to information missing from the training data required to perform the training operations.

Other types of recommendations or informational outputs may be provided in connection with the method 1100. These may include, generating one or more recommendations to obtain additional portions of the training data required to perform the training operations, and outputting the one or more recommendations in the user interface display. For instance, the one or more recommendations may include one or more recommendations for selection of one or more neurostimulation programs, one or more recommendations for an amount of usage of one or more neurostimulation programs, or both. Recommendations or metrics may be based on the ongoing collection of user feedback data, device data, or some combination.

The method 1100 continues at 1110 by optionally providing the training data to data processing system to perform training of a programming model (e.g., a model which selects a program or set of programming settings referred to herein as a programming selection model). Such training operations on the programming model can enable an updated set of parameters for the neurostimulation device. As noted above, the training data may be provided from user feedback data, device data, or some combination.

In further examples, the method 1100 continues at 1112 by optionally performing neurostimulation device programming based on the trained programming model. For instance, such programming (e.g., closed-loop programming) can be used to cause a change to neurostimulation programming settings on the neurostimulation device, as the change to the neurostimulation programming settings controls one or more of: pulse patterns, pulse shapes, a spatial location of pulses, waveform shapes, or a spatial location of waveform shapes, of modulated energy provided with a plurality of leads of the neurostimulation device.

Further steps of data processing such as data imputation may be performed in one or more intermediate steps between evaluating the patient feedback data and generating programming values (such as closed loop recommendations). In addition to data imputation that is used for determining programming values and that enables closed-loop processing, data imputation may be used to identify what information to indicate (recommend or suggest) to the patient about the amount of data collected, and what additional data is needed to train the model.

In specific training examples, the programming selection model is trained and used to provide closed-loop programming for the neurostimulation device, and the programming selection model is trained at a remote data service based on the training data that is collected with the operations 1102-1110 above. For instance, the training operations on the programming selection model may be performed at a remote data service, and the updated set of parameters produced by the training operations are generated by the programming selection model with one or more of: a new program for the neurostimulation device, a modified program for the neurostimulation device, or a modified schedule for use of multiple programs on the neurostimulation device.

In specific examples, the method 1100 may be used to determine or compare (track) usage of multiple neurostimulation programs for a particular patient, with such multiple neurostimulation programs being associated with respective sets of parameters. In this setting, evaluating the patient feedback data includes evaluating the usage of each of the multiple neurostimulation programs and the patient feedback data associated with each of the multiple neurostimulation programs. Also, calculating the amount of training data required to perform training operations on the programming selection model can also be based on the usage of each of the multiple neurostimulation programs and the patient feedback data (and optionally, device data) associated with each of the multiple neurostimulation programs.

In further examples, other types of treatment or recommendation processes may be provided as a result of the training of a programming model or the neurostimulation device programming. These may include determining disease treatment options or determining chronic pain treatment options (e.g., physical therapy, exercise, activities, medications, etc.). Other types of computer-determined or clinician-directed actions may be provided, consistent with the examples above.

FIG. 12 illustrates, by way of example, a block diagram of an embodiment of a system 1200 (e.g., a computing system) for performing analysis of patient feedback data (e.g., patient input data collected as training data in a user interface 1210) in connection with the data processing operations discussed above. The system 1200 may be integrated with or coupled to a computing device, a remote control device, patient programmer device, clinician programmer device, program modeling system, or other external device, deployed with neurostimulation treatment. In some examples, the system 1200 may be a networked device (server) connected via a network (or combination of networks) which communicates to one or more devices (clients) using a communication interface 1208 (e.g., communication hardware which implements software network interfaces and services). The network may include local, short-range, or long-range networks, such as Bluetooth, cellular, IEEE 802.11 (Wi-Fi), or other wired or wireless networks.

The system 1200 includes a processor 1202 and a memory 1204, which can be optionally included as part of user input/output data processing circuitry 1206. The processor 1202 may be any single processor or group of processors that act cooperatively. The memory 1204 may be any type of memory, including volatile or non-volatile memory. The memory 1204 may include instructions, which when executed by the processor 1202, cause the processor 1202 to implement data processing, or to enable other features of the user input/output data processing circuitry 1206. Thus, electronic operations in the system 1200 may be performed by the processor 1202 or the circuitry 1206.

For example, the processor 1202 or circuitry 1206 may implement any of the features of the method 1100 (such as operations 1102-1112) to obtain and process patient feedback data, to generate user interface displays, and to provide training data for training of a programming selection model. It will be understood that the processor 1202 or circuitry 1206 may also implement aspects of the logic and processing described above, for use in various forms of closed-loop and partially-closed-loop device programming or related device actions.

FIG. 13 illustrates, by way of example, a block diagram of an embodiment of a system 1300 (e.g., a computing system) implementing neurostimulation programming circuitry 1306 to cause programming of an implantable electrical neurostimulation device, for accomplishing the therapy objectives in a human subject based on a trained closed-loop programming model as discussed herein. The system 1300 may be operated by a clinician, a patient, a caregiver, a medical facility, a research institution, a medical device manufacturer or distributor, and embodied in a number of different computing platforms. The system 1300 may be a remote control device, patient programmer device, program modeling system, or other external device, including a regulated device used to directly implement programming commands and modification with a neurostimulation device. In some examples, the system 1300 may be a networked device connected via a network (or combination of networks) to a computing system operating a user interface computing system using a communication interface 1308. The network may include local, short-range, or long-range networks, such as Bluetooth, cellular, IEEE 802.11 (Wi-Fi), or other wired or wireless networks.

The system 1300 includes a processor 1302 and a memory 1304, which can be optionally included as part of neurostimulation programming circuitry 1306. The processor 1302 may be any single processor or group of processors that act cooperatively. The memory 1304 may be any type of memory, including volatile or non-volatile memory. The memory 1304 may include instructions, which when executed by the processor 1302, cause the processor 1302 to implement the features of the neurostimulation programming circuitry 1306. Thus, the electronic operations in the system 1300 may be performed by the processor 1302 or the circuitry 1306.

The processor 1302 or circuitry 1306 may directly or indirectly implement neurostimulation operations associated with the method 1100, including the use of neurostimulation device programming based on a trained programming model (operation 1112). The processor 1302 or circuitry 1306 may further provide data and commands to assist the processing and implementation of the programming using communication interface 1308 or a neurostimulation device interface 1310. It will be understood that the processor 1302 or circuitry 1306 may also implement other aspects of the device data processing or device programming functionality described above.

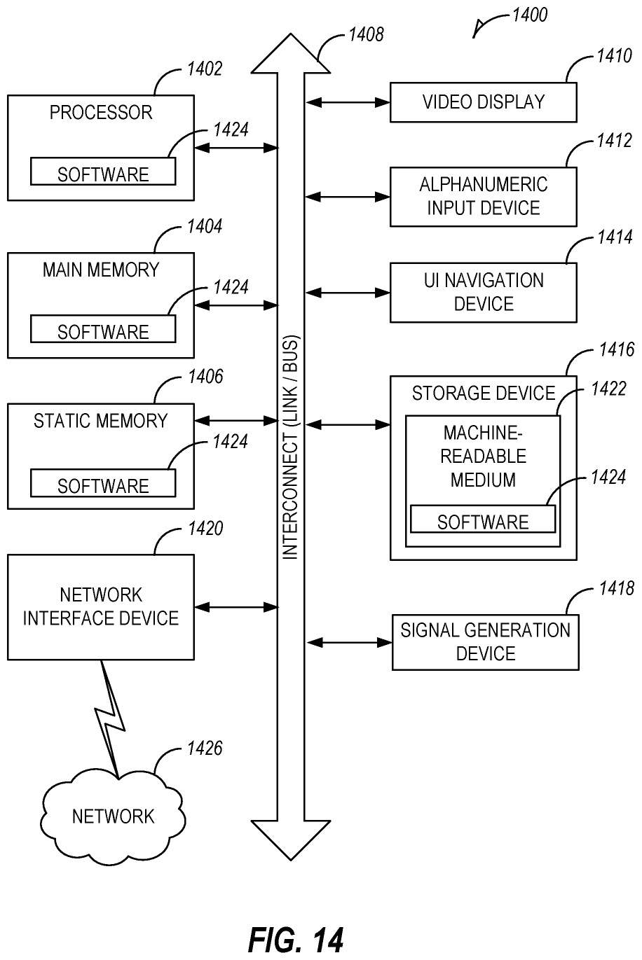

FIG. 14 is a block diagram illustrating a machine in the example form of a computer system 1400, within which a set or sequence of instructions may be executed to cause the machine to perform any one of the methodologies discussed herein, according to an example embodiment. In alternative embodiments, the machine operates as a standalone device or may be connected (e.g., networked) to other machines. In a networked deployment, the machine may operate in the capacity of either a server or a client machine in server-client network environments, or it may act as a peer machine in peer-to-peer (or distributed) network environments. The machine may be a personal computer (PC), a tablet PC, a hybrid tablet, a personal digital assistant (PDA), a mobile telephone, an implantable pulse generator (IPG), an external remote control (RC), a User's Programmer (CP), or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein. Similarly, the term “processor-based system” shall be taken to include any set of one or more machines that are controlled by or operated by a processor (e.g., a computer) to individually or jointly execute instructions to perform any one or more of the methodologies discussed herein.

Example computer system 1400 includes at least one processor 1402 (e.g., a central processing unit (CPU), a graphics processing unit (GPU) or both, processor cores, compute nodes, etc.), a main memory 1404 and a static memory 1406, which communicate with each other via a link 1408 (e.g., bus). The computer system 1400 may further include a video display unit 1410, an alphanumeric input device 1412 (e.g., a keyboard), and a user interface (UI) navigation device 1414 (e.g., a mouse). In one embodiment, the video display unit 1410, input device 1412 and UI navigation device 1414 are incorporated into a touch screen display. The computer system 1400 may additionally include a storage device 1416 (e.g., a drive unit), a signal generation device 1418 (e.g., a speaker), a network interface device 1420, and one or more sensors (not shown), such as a global positioning system (GPS) sensor, compass, accelerometer, or other sensor. It will be understood that other forms of machines or apparatuses (such as PIG, RC, CP devices, and the like) that are capable of implementing the methodologies discussed in this disclosure may not incorporate or utilize every component depicted in FIG. 14 (such as a GPU, video display unit, keyboard, etc.).

The storage device 1416 includes a machine-readable medium 1422 on which is stored one or more sets of data structures and instructions 1424 (e.g., software) embodying or utilized by any one or more of the methodologies or functions described herein. The instructions 1424 may also reside, completely or at least partially, within the main memory 1404, static memory 1406, and/or within the processor 1402 during execution thereof by the computer system 1400, with the main memory 1404, static memory 1406, and the processor 1402 also constituting machine-readable media.

While the machine-readable medium 1422 is illustrated in an example embodiment to be a single medium, the term “machine-readable medium” may include a single medium or multiple media (e.g., a centralized or distributed database, and/or associated caches and servers) that store the one or more instructions 1424. The term “machine-readable medium” shall also be taken to include any tangible (e.g., non-transitory) medium that is capable of storing, encoding or carrying instructions for execution by the machine and that cause the machine to perform any one or more of the methodologies of the present disclosure or that is capable of storing, encoding or carrying data structures utilized by or associated with such instructions. The term “machine-readable medium” shall accordingly be taken to include, but not be limited to, solid-state memories, and optical and magnetic media. Specific examples of machine-readable media include non-volatile memory, including but not limited to, by way of example, semiconductor memory devices (e.g., electrically programmable read-only memory (EPROM), electrically erasable programmable read-only memory (EEPROM)) and flash memory devices; magnetic disks such as internal hard disks and removable disks; magneto-optical disks; and CD-ROM and DVD-ROM disks.

The instructions 1424 may further be transmitted or received over a communications network 1426 using a transmission medium via the network interface device 1420 utilizing any one of a number of well-known transfer protocols (e.g., HTTP). Examples of communication networks include a local area network (LAN), a wide area network (WAN), the Internet, mobile telephone networks, plain old telephone (POTS) networks, and wireless data networks (e.g., Wi-Fi, 3G, and 4G LTE/LTE-A or 5G networks). The term “transmission medium” shall be taken to include any intangible medium that is capable of storing, encoding, or carrying instructions for execution by the machine, and includes digital or analog communications signals or other intangible medium to facilitate communication of such software.

The above detailed description is intended to be illustrative, and not restrictive. The scope of the disclosure should, therefore, be determined with references to the appended claims, along with the full scope of equivalents to which such claims are entitled.

What is claimed is:

1. A device to analyze data for neurostimulation programming, the device comprising:
one or more processors; and
one or more memory devices comprising instructions, which when executed by the one or more processors, cause the one or more processors to:
determine usage of a particular neurostimulation treatment, wherein the neurostimulation treatment is associated with a set of parameters used by a neurostimulation device to deliver neurostimulation to a human patient;
evaluate patient feedback data associated with the usage of the neurostimulation treatment, wherein the patient feedback data is obtained from the human patient; and
calculate, based on the patient feedback data, an amount of training data required to perform training operations on a programming selection model, wherein the training data is provided at least in part from the patient feedback data, and wherein the training operations on the programming selection model enable an updated set of parameters for the neurostimulation device.

2. The device of claim 1, wherein the instructions further cause the one or more processors to:
generate a user interface display to include one or more metrics that correspond to the amount of training data required to perform the training operations on the programming selection model.

3. The device of claim 2, wherein the user interface display further includes one or more metrics that correspond to an amount of training data available to perform the training operations on the programming selection model, and one or more values that correspond to information missing from the training data required to perform the training operations.

4. The device of claim 2, wherein the instructions further cause the one or more processors to:
generate one or more recommendations to obtain additional portions of the training data required to perform the training operations; and
output the one or more recommendations in the user interface display.

5. The device of claim 4, wherein the one or more recommendations includes one or more of:
one or more recommendations for selection of one or more neurostimulation programs; or
one or more recommendations for an amount of usage of one or more neurostimulation programs.

6. The device of claim 1, wherein the instructions further cause the one or more processors to:
evaluate device data associated with the usage of a particular neurostimulation program used to provide the neurostimulation treatment, wherein the device data is obtained from a wearable device or from the neurostimulation device, and wherein the training data is further provided from the device data;
wherein operations to calculate the amount of training data required to perform the training operations are further based on the device data; and
wherein operations to determine the usage of the neurostimulation treatment are further based on evaluating program usage as indicated by the device data.

7. The device of claim 6, wherein the patient feedback data relates to one or more of: sleep, pain, movement, fatigue or restfulness, alertness, emotional state, medication state, mobility, or activity, in connection with use of the neurostimulation treatment, and wherein the patient feedback data is obtained from the human patient using one or more: questionnaires, surveys, text entries, or voice inputs.

8. The device of claim 1, wherein the instructions further cause the one or more processors to:
identify, based on the calculated amount of training data required to perform training operations, availability for retraining of the programming selection model; and
generate a user interface display that includes a user-selectable option to cause the retraining of the programming selection model.

9. The device of claim 1, wherein the instructions further cause the one or more processors to:
determine usage of multiple neurostimulation programs, wherein the multiple neurostimulation programs are associated with respective sets of parameters;
wherein to evaluate the patient feedback data includes to evaluate the usage of each of the multiple neurostimulation programs and the patient feedback data associated with each of the multiple neurostimulation programs; and
wherein to calculate the amount of training data required to perform training operations on the programming selection model is further based on the usage of each of the multiple neurostimulation programs and the patient feedback data associated with each of the multiple neurostimulation programs.

10. The device of claim 1, wherein the programming selection model is used to provide closed-loop programming for the neurostimulation device;
wherein the closed-loop programming causes a change to neurostimulation programming settings on the neurostimulation device;
wherein the change to the neurostimulation programming settings controls one or more of: pulse patterns, pulse shapes, a spatial location of pulses, waveform shapes, or a spatial location of waveform shapes, of modulated energy provided with a plurality of leads of the neurostimulation device; and
wherein the programming selection model is trained at a remote data service based on the training data.

11. A method for analyzing data for neurostimulation programming, comprising:
determining usage of a particular neurostimulation treatment, wherein the neurostimulation treatment is associated with a set of parameters used by a neurostimulation device to deliver neurostimulation to a human patient;
evaluating patient feedback data associated with the usage of the neurostimulation treatment, wherein the patient feedback data is obtained from the human patient; and
calculating, based on the patient feedback data, an amount of training data required to perform training operations on a programming selection model, wherein the training data is provided at least in part from the patient feedback data, and wherein the training operations on the programming selection model enable an updated set of parameters for the neurostimulation device.

12. The method of claim 11, further comprising:
generating a user interface display to include one or more metrics that correspond to the amount of training data required to perform the training operations on the programming selection model.

13. The method of claim 12, wherein the user interface display further includes one or more metrics that correspond to an amount of training data available to perform the training operations on the programming selection model, and one or more values that correspond to information missing from the training data required to perform the training operations.

14. The method of claim 12, further comprising:
generating one or more recommendations to obtain additional portions of the training data required to perform the training operations; and
outputting the one or more recommendations in the user interface display.

15. The method of claim 14, wherein the one or more recommendations includes one or more of:
one or more recommendations for selection of one or more neurostimulation programs; or
one or more recommendations for an amount of usage of one or more neurostimulation programs.

16. The method of claim 11, further comprising:
evaluating device data associated with the usage of a particular neurostimulation program used to provide the neurostimulation treatment, wherein the device data is obtained from a wearable device or from the neurostimulation device, and wherein the training data is further provided from the device data;
wherein calculating the amount of training data required to perform the training operations are further based on the device data; and
wherein determining the usage of the neurostimulation treatment are further based on evaluating program usage as indicated by the device data.

17. The method of claim 16, wherein the patient feedback data relates to one or more of: sleep, pain, movement, fatigue or restfulness, alertness, emotional state, medication state, mobility, or activity, in connection with use of the neurostimulation treatment, and wherein the patient feedback data is obtained from the human patient using one or more: questionnaires, surveys, text entries, or voice inputs.

18. The method of claim 11, further comprising:
identifying, based on the calculated amount of training data required to perform training operations, availability for retraining of the programming selection model; and
generating a user interface display that includes a user-selectable option to cause the retraining of the programming selection model.

19. The method of claim 11, further comprising:
determining usage of multiple neurostimulation programs, wherein the multiple neurostimulation programs are associated with respective sets of parameters;
wherein evaluating the patient feedback data includes evaluating the usage of each of the multiple neurostimulation programs and the patient feedback data associated with each of the multiple neurostimulation programs; and
wherein calculating the amount of training data required to perform training operations on the programming selection model is further based on the usage of each of the multiple neurostimulation programs and the patient feedback data associated with each of the multiple neurostimulation programs.

20. The method of claim 11, wherein the programming selection model is used to provide closed-loop programming for the neurostimulation device;
wherein the closed-loop programming causes a change to neurostimulation programming settings on the neurostimulation device;
wherein the change to the neurostimulation programming settings controls one or more of: pulse patterns, pulse shapes, a spatial location of pulses, waveform shapes, or a spatial location of waveform shapes, of modulated energy provided with a plurality of leads of the neurostimulation device; and
wherein the programming selection model is trained at a remote data service based on the training data.
